(19) United States
(12) Patent Application Publication
(10) Pub. No.: US 2024/0069006 A1
(43) Pub. Date: Feb. 29, 2024

(54) CONDUCTIVE AND TRANSPARENT INTERCONNECTION STRUCTURE, ASSOCIATED MANUFACTURING METHOD AND SYSTEM

(71) Applicant: COMMISSARIAT À L’ENERGIE ATOMIQUE ET AUX ENERGIES ALTERNATIVES, PARIS (FR)

(72) Inventors: Benoit RACINE, GRENOBLE CEDEX 09 (FR); Olivier HAON, GRENOBLE CEDEX 09 (FR)

(21) Appl. No.: 18/457,549

(22) Filed: Aug. 29, 2023

(30) Foreign Application Priority Data
Aug. 29, 2022 (FR) 2208619

(51) Int. Cl. G01N 33/483; G01N 21/17

(52) U.S. Cl. CPC G01N 33/4833; G01N 21/1717; G01N 2021/1721

(57) ABSTRACT

An interconnection structure includes a substrate formed by a first electrically insulating and optically transparent material, the substrate including a first face and an opposite second face, the first face defining a plane of the substrate, and a plurality of transparent electrodes, wherein the transparent electrodes pass through the substrate from the first face to the second face of the substrate in parallel to each other, and are electrically insulated from each other by the first electrically insulating and optically transparent material.

### CROSS-REFERENCE TO RELATED APPLICATIONS

This application claims priority to French Patent Application No. 2208619, filed Aug. 29, 2022, the entire content of which is incorporated herein by reference in its entirety.

### FIELD

The field of the invention is that of electrical, optical and mechanical interconnection between an electronic component and a sample to be analysed, for example a biological sample such as neuronal cells in culture in a microfluidic cell.

The invention finds an application in the fields of biology and health, in particular in the field of organs-on-chips.

### BACKGROUND

Organs-on-chips are miniaturised technological platforms in which cells are cultured in vitro and combined with microfluidic and microelectronic technologies and sensors, which may be electronic and/or optical sensors. Organs-on-chips make it possible to reproduce and study the way organs function as closely as possible to how they function in the human body.

Applied to neuronal cells (or “neurons”), organs-on-chips offer the possibility of understanding some neurodegenerative diseases, such as Parkinson's disease or Alzheimer's disease, and of testing in vitro treatments introduced via the microfluidic circuit.

Real-time measurement of the electrical activity of neuronal cells is one of the pathways to understanding these diseases. The electrical activity of neuronal cells results in the emission of action potentials in the order of tens of millivolts, for example 70 mV. These action potentials can be received either directly by an electronic sensor onto which the neuronal cells are deposited, or via an interfacing device disposed between the neuronal cells and an electronic sensor.

To measure action potentials of a neuronal cell directly, it is known to bring a neuronal cell into contact with a surface covered with a thin dielectric and connected to the gate of an MOS (“Metal Oxide Semiconductor”) transistor. When the neuronal cell is electrically stimulated, it emits action potentials that are capacitively coupled to the transistor. In other words, the transistor acts as a capacitive voltage sensor capable of recording the electrical activity of an individual cell.

An electronic sensor called a “Multi Transistor Array” has been designed on this basis. It includes a plurality of MOS transistors connected in series to form an array or matrix of transistors.

The size of the transistors and the pitch of the transistor array ensure that each neuronal cell is electrically measured individually, whatever its position on the surface of the matrix. The array records the electrical activity of the cells in parallel. Such a sensor therefore functions as an electrophysiological imaging device whose spatial resolution is at the scale of the neuronal cell.

For example, such a matrix of MOS transistors including 16 384 transistors arranged in series to form an analysis surface area of 1 mm² has been developed. Such a device makes it possible to grow or culture neuronal cells on the transistors and to measure electrical activity of these cells.

The drawback of this type of sensor is that it comprises transistors formed on silicon substrates, which are therefore opaque to visible light. This is a major restriction when it is desired to observe or measure activity of neuronal cells optically, that is, by microscopy or any other imaging mode using visible light, in order to benefit from the complementarity between optical and electrical characterisation methods. More precisely, because of the opacity of the transistors, the only optical imaging modality possible is the reflection (or “epi”) modality, which consists in measuring the signal reflected by the sample. This modality limits the type of optical signals that can be measured on the cells.

To overcome this, it is possible to use an interconnection device that is completely transparent to visible light. In general, such a device does not comprise transistors but conductive zones—called electrodes—electrically and passively connecting input contact points and output contact points. Thus, when neuronal cells are disposed onto the input contact points, their electrical activity can be collected and transmitted to the output contacts, then processed by the outer sensors connected to the output contacts.

Such a device is described in patent EP2245454B1 for measuring electrical activity of a biological sample. It is an array of passive microelectrodes having transparency and biocompatibility characteristics.

The microelectrode array is said to be passive in the sense that it comprises microelectrode circuit arrangements used solely for the transduction of bioelectric signals, that is, without any other preprocessing. The materials used for the substrate and the microelectrode array are transparent polymers, which allows optical observation of the sample through said array.

However, such an array does not allow an image of the electrical activity produced by the sample to be transmitted directly and simply. Indeed, it is difficult to establish a correspondence between an output signal delivered by an output contact and the part of the sample that is electrically active. To achieve this, it is necessary to connect the output contacts by a wire link (or similar) and to take account of the routing plan giving the correspondence between each output contact and the position of the measurement electrode in the array.

There is therefore a need for a transparent interconnection device allowing the electrical activity of the sample to be imaged simply and directly.

This need exists for the study of a neuronal cell sample, and more generally for the study of samples comprising at least one source capable of generating an electrical signal.

### SUMMARY

An aspect of the invention offers a solution to the problem previously discussed by providing a transparent interconnection structure with a through-electrode structure making it possible to electrically connect an output contact to a single input contact while keeping the spatial information of the input contact, in particular its position relative to the other contacts.

A first aspect of the invention relates to an interconnection structure comprising:
a substrate formed by a first electrically insulating and optically transparent material, the substrate comprising a first face and an opposite second face, the first face defining a plane of the substrate;
a plurality of transparent electrodes;
the interconnection structure being remarkable in that the transparent electrodes pass through the substrate from the first face to the second face of the substrate in parallel to each other, and are electrically insulated from each other by the first electrically insulating and optically transparent material.

Thus, a plurality of transparent electrodes, called through-electrodes, pass through the substrate between the first and second faces, that is, in the direction of its thickness, while being electrically insulated from each other by the substrate.

The ends of the through-electrodes form, on or in the first face and on or in the second face of the substrate, electrical contacts that can receive or output a voltage. Each electrical contact on the first face is connected to a single electrical contact on the second face, while keeping the spatial information relating to the electrical contact on the first face. By spatial information, it is meant the position of the contact relative to the other contacts on the face. In other words, two connected electrical contacts each occupy substantially the same position on their face relative to the other contacts. There is therefore a spatial correspondence between the electrical contacts on the first face and the electrical contacts on the second face of the substrate, which enables an electrical image formed on the first face to be transmitted faithfully and directly to the second face, that is, without the need for processing, an outer electronic device or a wire connection.

Furthermore, when the first face of the substrate is lit by an incident light wave in the visible range, it is possible to detect a light wave transmitted directly facing the electrical contacts of the second face, without the structure of the electrodes or the substrate significantly interfering with the detection. The electrodes and substrate are optically transparent, that is, they have an optical transmission coefficient greater than 85% for at least one wavelength in the 400-800 nm spectral band.

The interconnection structure therefore enables an electrical image and a light wave transmitted through the substrate to be transmitted simultaneously and superimposed. In other words, the interconnection structure according to an aspect of the invention makes it possible to combine optical and electrical imaging and thus to benefit from their complementarity.

Beneficially, each transparent electrode comprises a first portion disposed on the first surface of the substrate.

The first portion makes it possible to obtain a compact electrode end, free from any cavity, void or hollow, these irregularities being able to degrade both the conductivity of the electrode and the quality of the electrical coupling of the electrode with a voltage source, typically a biological sample.

Thus, the first portion allows better electrical contact between the electrode and the voltage source.

Finally, the first portion allows the dimensions of the electrode to be extended laterally, so as to increase the electrical contact surface area and/or adapt the electrical spatial resolution.

Beneficially, the first portion has dimensions of between 6 μm and 55 μm in the plane of the substrate, and a thickness of between 5 nm and 100 nm.

Thus, the spatial resolution is micrometric, for example in the order of 10 μm.

Beneficially, each transparent electrode further comprises a second portion disposed on the second face of the substrate facing the first portion.

The second portion makes it possible to improve the conductivity of the electrode by obtaining a compact second electrode end, free from any cavity, void or hollow. The second portion also makes it possible to improve the electrical contact between the electrode and a voltage receiver, typically an electronic sensor or an electro-optical device.

Beneficially, the second portion has dimensions of between 6 μm and 55 μm in a plane parallel to the plane of the substrate, and a thickness of between 5 nm and 100 nm.

Beneficially, the transparent electrodes are symmetrical relative to a plane parallel to the plane of the substrate.

This improves the fidelity of the electrical image transmitted by the interconnection structure. When the interconnection structure comprises a first and a second portion, these have identical cross-sections (in a plane parallel to the substrate). By identical cross-sections, it is meant that the cross-section of the first portion and the cross-section of the second portion are the same shape (circular, square, rectangular, hexagonal, etc.) and the same dimensions. Thus, there is a spatial correspondence, both in position and in dimensions, between the electrical contacts of the first face and the electrical contacts of the second face.

In an embodiment, each transparent electrode comprises a hollow cylindrical portion extending from the first face to the second face of the substrate, the interconnection structure further comprising a transparent core disposed inside the hollow cylindrical portion of each transparent electrode.

In an embodiment, the hollow cylindrical portion has, in a plane parallel to the plane of the substrate, external dimensions of between 5 μm and 50 μm, and comprises a wall with a thickness of between 5 nm and 100 nm.

In a first embodiment, the transparent core is formed by an electrically conductive and optically transparent material.

This characteristic makes it possible to optimise the conductivity of the electrode while guaranteeing the transparency of the electrode. In this first embodiment, the transparent core forms part of the electrode

In a second embodiment, the transparent core is formed by a second electrically insulating and optically transparent material.

This characteristic makes it possible to optimise the transparency of the electrode while guaranteeing satisfactory conductivity of the electrode. In this second embodiment, the transparent core does not form part of the electrode.

The interconnection structure according to the first aspect of the invention may also have one or more of the following characteristics, considered individually or according to any technically possible combination.

The first electrically insulating and optically transparent material may be a glass, a resin, an organo-mineral polymer or an expanded polystyrene.

The substrate may have a thickness between the first face and the second face of the substrate of between 50 μm and 300 μm.

The interconnection structure can thus have sufficient mechanical rigidity to receive a biological sample encapsulated in a fluidic or micro-fluidic chamber.

The substrate may have dimensions of between 2.5 cm and 10 cm in a plane parallel to the plane of the substrate.

Such a substrate makes it possible to obtain one or more large active surface areas, that is, in the order of one cm². By active surface area, it is meant the surface area covered by several transparent through-electrodes relative to the surface of the substrate.

The transparent electrodes can be spaced apart from each other inside the substrate by an edge-to-edge distance of between 5 μm and 30 μm.

Thus, the spatial resolution is micrometric.

Beneficially, the transparent electrodes are identical in size and shape and have, in the plane of the substrate, a first repeat pitch in a first direction and a second repeat pitch in a second direction intersecting the first direction.

The first repeat pitch and the second repeat pitch may or may not be identical.

The transparent through-electrodes thus form a matrix array, which facilitates the manufacture and use of the interconnection structure. For example, the interconnection structure is made directly compatible with matrix sensors.

The transparent electrodes may be formed by a transparent conductive polymer or a transparent conductive metal oxide such as tin dioxide.

Tin dioxide (SnO₂) has a particular benefit during manufacture as it is compatible with a conformal deposition method, which is not the case for a transparent conductive material such as indium tin oxide (ITO).

A second aspect of the invention relates to a method for manufacturing an interconnection structure, comprising the following steps of:
providing a substrate formed by a first electrically insulating and optically transparent material, the substrate comprising a first face and an opposite second face, the first face defining a plane of the substrate;
arranging, in the substrate, a plurality of cavities, called through-cavities, passing through the substrate between the first face and the second face of the substrate in parallel to each other, and being spaced apart from each other,
forming a plurality of transparent electrodes in the through-cavities, the transparent electrodes passing through the substrate from the first face to the second face of the substrate in parallel to each other, and being electrically insulated from each other by the first electrically insulating and optically transparent material.

In an embodiment, the step of forming the plurality of transparent electrodes comprises the following steps of:
depositing a first layer of electrically conductive and optically transparent material onto:
   the inner surface of each of the through-cavities,
   the first face of the substrate, and
   the second face of the substrate;
filling the through-cavities with transparent material and form an extra thickness of transparent material on the first and second faces of the substrate;
removing the extra thickness of transparent material as well as portions of the first layer of electrically conductive and optically transparent material deposited onto the first face and the second face of the substrate.

In an embodiment, the step of depositing the first layer of electrically conductive and optically transparent material is carried out with a conformal deposition method such as the Atomic Layer Deposition (ALD) method.

In an embodiment, the step of forming the transparent electrodes comprises an additional step of forming, for each of the through-electrodes formed, a first transparent electrode portion on the first face of the substrate and a second transparent electrode portion on the second face of the substrate, the additional step comprising the following sub-steps of:
depositing a second layer of electrically conductive and optically transparent material onto the first face and the second face of the substrate,
etching the second layer of electrically conductive and optically transparent material in regions of the first and second faces of the substrate, the regions being located around the transparent electrodes formed.

Beneficially, the step of depositing the second layer of electrically conductive and optically transparent material is carried out using a deposition method compatible with a transparent conductive oxide (TCO).

A third aspect of the invention is a system for analysing a sample comprising:
a light source producing a light wave, called an incident light wave,
an interconnection structure according to the first aspect of the invention, the first face of the substrate being disposed facing the light source and being intended to receive a sample comprising at least one source capable of producing an electrical signal, called the electrical signal of the sample, the electrical signal of the sample being transmitted from the first face of the substrate to the second face of the substrate by the through-electrode located facing the source,
an electro-optical device disposed facing the second face of the substrate,
the electro-optical device having a plurality of control electrodes disposed facing the plurality of through-electrodes of the interconnection structure, each control electrode of the plurality of control electrodes being connected to a through-electrode of the plurality of through-electrodes, so that the electro-optical device modulates the light wave transmitted by the sample under the effect of the electrical signal of the sample.

According to an embodiment of the third aspect of the invention, the electro-optical device is a liquid crystal cell.

It is thus possible to characterise the electrical activity of the sample indirectly by using the complementarity of the optical and electrical imaging modes. This aspect of the invention has a particular benefit for the study of the electrical activity of neuronal cells in culture, and more generally for making organs-on-chips.

The invention and its different applications will be better understood upon reading the following description and upon examining the accompanying figures.

### BRIEF DESCRIPTION OF THE FIGURES

The figures are set forth by way of indicating and in no way limiting purposes of the invention.

FIG. 1 schematically represents, in a top view, a first embodiment of the interconnection structure;

FIG. 2 is a cross-sectional view of the interconnection structure according to the first embodiment;

FIG. 3 is a cross-sectional view of the interconnection structure according to a second embodiment;

FIG. 4 is a block diagram illustrating the sequence of steps in the method for manufacturing the interconnection structure;

FIG. 5 is a block diagram illustrating an implementation of the third step of the manufacturing method;

FIG. 6 is a block diagram illustrating an implementation of the fourth sub-step of the third step of the manufacturing method;

FIGS. 7A to 7G represent cross-sectional views of the steps or sub-steps of manufacturing the interconnection structure;

FIG. 8 schematically represents a system for analysing a sample, comprising the interconnection structure;

FIG. 9 is an enlarged view of FIG. 8 centred on the interconnection structure in the analysis system and on a sample disposed on the interconnection structure.

### DETAILED DESCRIPTION

The figures are set forth by way of indicating and in no way limiting purposes of the invention.

Unless otherwise specified, a same element appearing in different figures has a unique reference.

In the description, the term “optically transparent” or “transparent” refers to any material or element that has an optical transmission coefficient greater than 85% for at least one wavelength in the spectral band extending in the visible range, that is, between 400 nm and 800 nm.

A first aspect of the invention relates to an optically transparent interconnection structure 1 comprising a plurality of through-electrodes 20.

The interconnection structure 1 is for example intended to interface between a biological sample and an electronic or electro-optical device for imaging the electrical and/or optical activity of the sample.

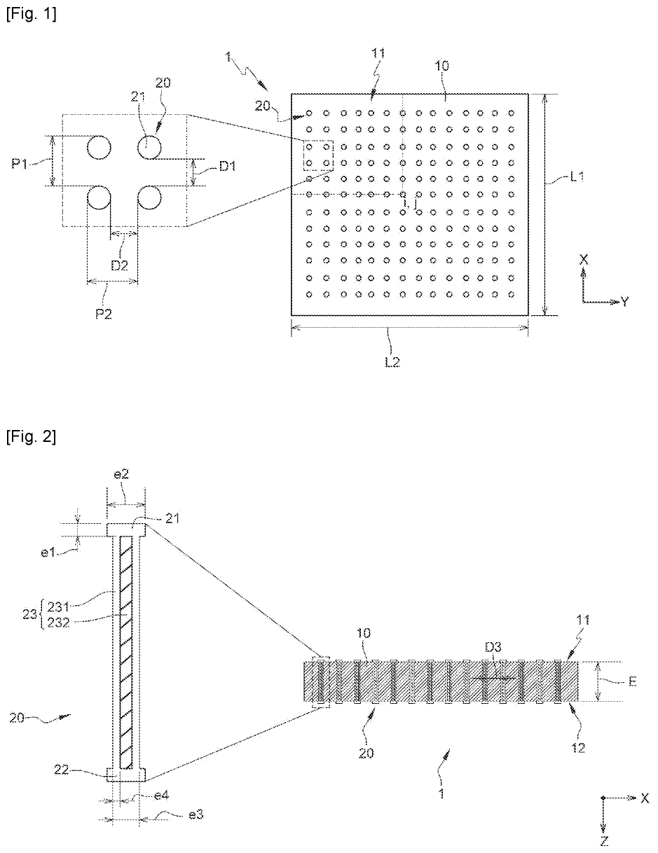

The interconnection structure 1 is described with reference to FIGS. 1 to 3, which schematically represent, in a top view and in a cross-sectional view, two embodiments of the interconnection structure 1. The top view (FIG. 1) is common to both embodiments.

With reference to FIGS. 1, 2 and 3, the interconnection structure 1 comprises an insulating transparent substrate 10 having a first face 11 and a second face 12 opposite to the first face 11, and a plurality of transparent through-electrodes 20. The plane defined by the first face 11 of the substrate 10 will be called the plane of the substrate.

In these particular embodiments, the first and second faces 11, 12 of the substrate 10 are interchangeable because the interconnection structure 1 is symmetrical along a plane parallel to the plane of the substrate.

In the following description, these two faces will be distinguished solely by the fact that the first face 11 of the substrate 10 is intended to receive the sample, the second face 12 then being intended to be connected to an electronic device or to an electro-optical device.

The terms “lateral” or “laterally” refer to the axes X and Y as represented in FIG. 1, these axes X and Y defining the plane of the substrate. Furthermore, “surface covered by the electrodes” or “active surface area” refers to the surface covered by the electrodes in the plane of the substrate.

Finally, the transparent through-electrodes 20 will be referred to more simply as “through-electrodes 20” or “electrodes 20”.

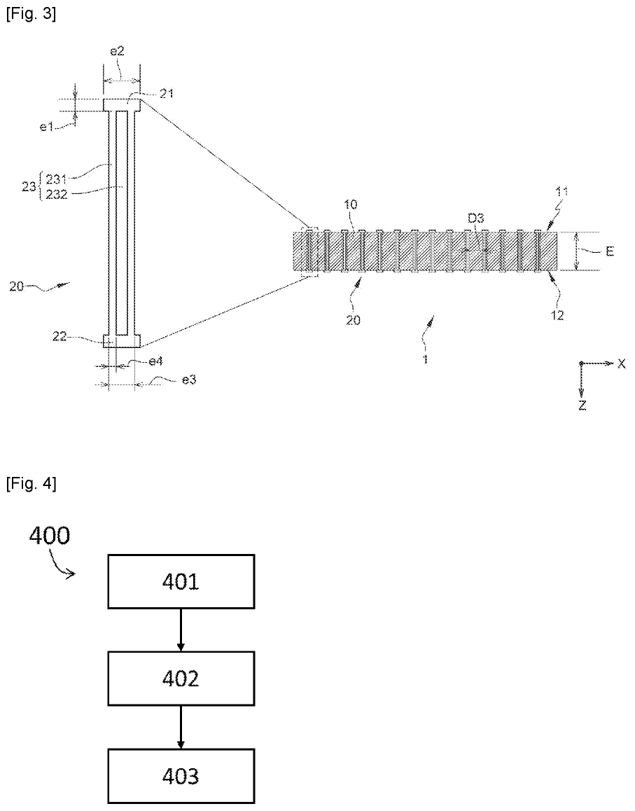

The substrate 10 has a thickness E between the first face 11 and the second face 12, and lateral dimensions L1 and L2. The lateral dimensions L1 and L2 may be between 2.5 cm and 10 cm, in order to obtain one or more active surface areas (corresponding to one or more groups of electrodes) in the order of one cm². The thickness E of the substrate 10 (and the thickness of any other element of the interconnection structure, unless otherwise stated) is measured perpendicularly to the plane of the substrate, along an axis Z represented in FIGS. 2 and 3.

The substrate 10 is formed by a first insulating and optically transparent material.

The substrate 10 is thus beneficially formed by a glass, a resin, an organo-mineral polymer such as polydimethylsiloxane (PDMS), expanded polystyrene or any other material which is insulating, optically transparent and offers sufficient mechanical rigidity to receive, on the first face 11, a fluid chamber enabling the sample to be held in a liquid medium.

The thickness E of the substrate 10 is chosen so as to give good mechanical rigidity to the substrate 10. Good mechanical rigidity means that the substrate 10 does not deform when it interacts with the sample. Depending on the material used, the thickness E of the substrate 10 can be between 50 μm and 300 μm.

For example, the substrate 10 is a glass with a thickness E of 300 μm and lateral dimensions L1=L2=2.4 cm, providing an active surface area of up to 5.76 cm².

The through-electrodes 20 are optically transparent conductive zones. By conductive zone, it is meant a zone formed by a conductive material enabling voltages ranging from a few millivolts to a few microvolts to be conducted substantially without losses. These orders of magnitude are typical of the voltages, called evoked potentials, produced by biological samples such as neuronal cells. The through-electrodes 20 are made of a material, polymer or oxide, which is both conductive and optically transparent, for example Poly(3,4-ethylenedioxythiophene) (PEDOT), indium tin oxide (ITO), tin dioxide (SnO₂), zinc oxide (ZnO) or aluminium-doped zinc oxide (AZO).

The through-electrodes 20 pass through the substrate over its entire thickness E, from the first face 11 to the second face 12 of the substrate 10 and are separated from each other by the first insulating and optically transparent material of the substrate 10. The first insulating and optically transparent material of the substrate 10 coats (laterally) the through-electrodes 20.

The through-electrodes 20 extend in parallel to each other, such as in a direction substantially perpendicular to the plane of the substrate, and for example along the axis Z. By substantially perpendicular direction, it is meant perpendicular to within a tolerance of a few degrees, for example 5 degrees (90°±5°).

Inside the substrate 10, the distance D3 between two neighbouring transparent electrodes 20, measured edge to edge, is for example between 5 μm and 30 μm. In FIGS. 2 and 3, the distance D3 is measured along the direction X.

FIGS. 2 and 3 also show a schematic cross-sectional view of an enlarged view of a through-electrode 20 according to a first embodiment and a second embodiment, respectively, of the interconnection structure 1.

Common to these two embodiments, the through-electrode 20 comprises, in addition to an internal portion 23 extending from the first face 11 to the second face 12 of the substrate 10, a first portion 21 disposed on the first face 11 of the substrate 10.

The through-electrode 20 may also comprise a second portion 22 disposed on the second face 12 of the substrate 10 facing the first portion 21. In this case, the internal portion 23 is extended by the first portion 21 and the second portion 22 which form the ends of the electrode 20. The first portion 21 and the second portion 22 are in direct contact with the internal portion so as to ensure electrical continuity of the electrode 20 from one face to the other of the substrate 10.

The first portion 21 is a thin layer of an electrically conductive and optically transparent material chosen from the electrically conductive and optically transparent materials mentioned previously. It may be the same material or a different material from that forming the internal portion 23 of the through-electrode 20.

A thin layer refers here to a layer with a thickness of between 5 nm and 100 nm. Thus, the thickness e1 of the first portion 21 is between 5 nm and 100 nm.

Laterally, the first portion 21 beneficially extends beyond the internal portion 23 of the electrode 20 (inside the substrate 10), thus increasing the contact surface area of the electrode 20 compared with an internal portion 23 alone. In an embodiment, the first portion 21 has lateral dimensions strictly greater than the lateral dimensions of the internal portion 23. Thus, the first portion 21 can have lateral dimensions of between 6 μm and 55 μm, for lateral dimensions of the internal portion 23 of between 5 μm and 50 μm. For example, in FIGS. 2 and 3, the first portion 21 occupies a circular surface with a diameter e2 of between 6 μm and 55 μm, while the internal portion 23 of the electrode 20 has a diameter e3 of between 5 μm and 50 μm.

The first portion 21 may have, in the plane of the substrate 11, a circular cross-section or another shape, for example a square, rectangular, hexagonal, etc. shape.

By conforming to the internal portion 23, the first portion 21 makes it possible to fill any hollows, holes or voids left on the surface of the internal portion 23 during manufacture of the electrodes. In this way, the first portion 21 helps to make a through-electrode 20 that is free from hollows and therefore offers better electrical conductivity. The first portion 21 further extends the surface area of the electrical contact while reducing the surface irregularities of this contact. Electrical coupling with the sample is thus improved.

The second portion 22 is similar to the first portion 21; the description, effects and benefits given for the first portion 21 therefore apply to the second portion 22. The conductive and optically transparent material chosen to form the second portion 22 may be the same as or different from the material chosen to form the first portion 21.

The first portion 21 and the second portion 22 may have, in planes parallel to the plane of the substrate, identical or, on the contrary, different cross-sections, for example circular, square, rectangular, hexagonal, etc., in shape.

In an embodiment, the electrode 20 is symmetrical relative to a plane parallel to the plane of the substrate. The first portion 21 and the second portion 22 then have an identical cross-section shape and dimensions. Thus, the second portion 22 has the effect of making the (input and output) electrical contacts of the through-electrode 20 symmetrical. The benefit is that the interconnection structure 1 can thus transmit an electrical image applied to the first face 11 of the substrate, to the second face 12 of the substrate 10 with improved spatial and electrical fidelity.

In an embodiment, the internal portion 23 of the through-electrode 20 is a hollow cylindrical portion 231 and the interconnection structure 1 further comprises a transparent core 232 disposed inside the hollow cylindrical portion 231 of each through-electrode 20.

The hollow cylindrical portion 231 comprises a wall consisting of a thin layer of an optically transparent and electrically conductive material, for example chosen from the optically transparent and electrically conductive materials mentioned previously. Even more beneficially, the optically transparent and electrically conductive material forming the hollow cylindrical portion 231 is a transparent metal oxide such as SnO₂. This material is beneficially the same as that used to make the first portion 21 and/or the second portion 22. The thickness e4 of the wall of the hollow cylindrical portion 232, measured in a plane parallel to the plane of the substrate, may be between 5 nm and 100 nm. The wall is connected to the first portion 21 and the second portion 22 so that the electrode is conductive. The cylindrical portion 231 may have a circular, hexagonal, rectangular, etc. cross-section.

In the first embodiment (see FIG. 2), the transparent core 232 is formed by an electrically conductive and optically transparent material. In an embodiment, the transparent core 232 is formed by a transparent polymer material, for example PEDOT. Indeed, PEDOT is one of the optically transparent materials having the best ionic and electronic conductivity. The conductivity of the through-electrode 20 is thus enhanced while preserving its transparency. This embodiment is particularly beneficial when the voltages involved are in the order of one μV. In this first embodiment, the transparent core 232 is considered to form part of the through-electrode 20, which then consists of a maximum of four elements, these four elements being the first portion 21, the second portion 22, the hollow cylindrical portion 231 and the transparent core 232.

In the second embodiment (see FIG. 3), the transparent core 232 is formed by a second electrically insulating and optically transparent material. In an embodiment, the transparent core 232 is formed by an optical grade adhesive, for example a Vitralit™ 6127, 6128 or equivalent adhesive, or an epoxy type resin. These types of materials have a transmission coefficient greater than that of conductive polymer materials such as PEDOT. The transparency of the through-electrode 20 is thus enhanced while preserving acceptable conductivity. This embodiment is particularly beneficial when the light waves transmitted by the interconnection structure 1 are of low luminance (less than 50 cd/m²). In this second embodiment, the transparent core 232 is not considered to form part of the through-electrode 20, which then consists of a maximum of three elements, these three elements being the first portion 21, the second portion 22 and the hollow cylindrical portion 231.

According to alternative embodiments not represented, the electrode 20 comprises only the internal portion 23, or only the internal portion 23 and the first portion 21, or only the internal portion 23 and the second portion 22.

With reference to FIG. 1, the transparent electrodes 20 are beneficially distributed along the two directions of the plane of the substrate.

On the first face 11 and the second face 12 of the substrate 10, the transparent electrodes 20 are spaced apart two by two by a distance D1, measured edge to edge, of between 1 μm and 28 μm along the direction X and by a distance D2, measured edge to edge, of between 1 μm and 28 μm along the direction Y. The distances D1 and D2 may be identical or different. The distances D1 and D2 are smaller than the distance D3 described previously because the first and second portions 21-22 have lateral dimensions greater than the lateral dimensions of the internal portion 23.

Beneficially, the through-electrodes 20 are all of identical shape and dimensions and have a first repeat pitch P1 in the first direction (here along the X axis) and a second repeat pitch P2 in a second direction intersecting the first direction (here along the Y axis perpendicular to the X axis).

In an embodiment, the repeat pitches P1 and P2 are micrometric, more precisely P1 and P2 are between 7 μm and 83 μm. The through-electrodes 20 are thus regularly spaced and arranged in rows and columns so as to form a high-density electrical matrix (>400 electrodes per mm²) with micrometric spatial resolution. The repeat pitches P1 and P2 are even more beneficially identical, avoiding spatial distortion of the transmitted voltages.

The through-electrodes 20 may not cover the entire surface of the substrate 10, and may be arranged in groups separated by portions of the substrate 10, or else be disposed more or less densely depending on the regions of the substrate 10 (not represented).

A second aspect of the invention relates to a method 400 for manufacturing the interconnection structure 1.

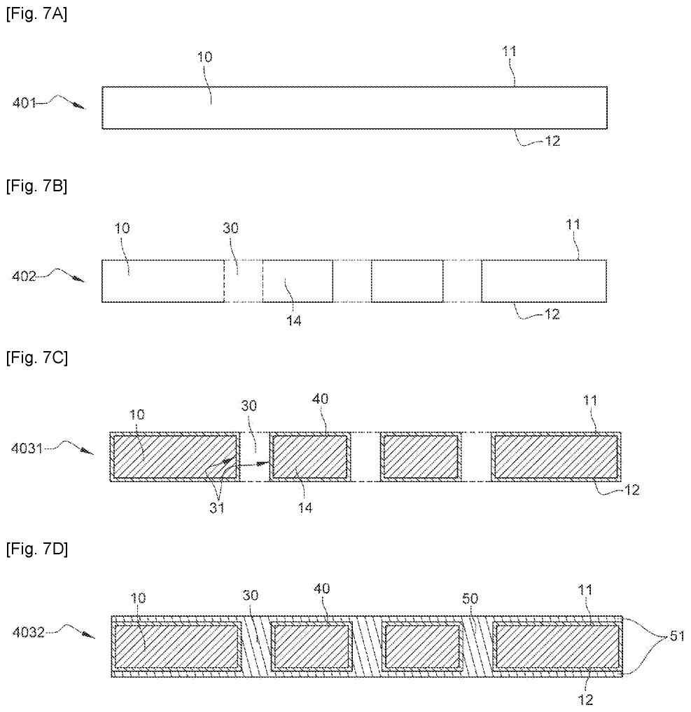

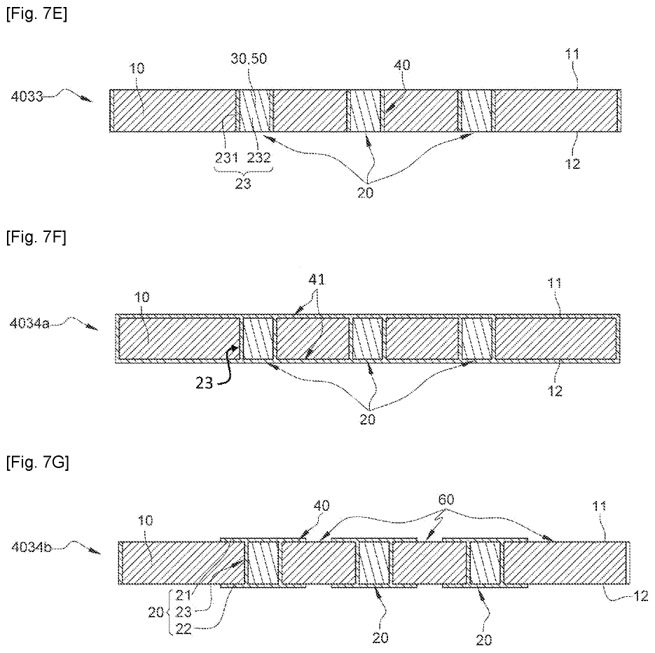

FIG. 4 is a block diagram illustrating the sequence of steps 401 to 403 of the method for manufacturing 400 the interconnection structure 1. These same steps 401 to 403 are illustrated by FIGS. 7A to 7C, by virtue of schematic cross-sectional views of the interconnection structure 1.

With reference to FIG. 7A, the first step 401 consists in providing the substrate 10 formed by the first electrically insulating and optically transparent material.

The second step 402 is illustrated in FIG. 7B and consists in arranging, in the substrate, a plurality of cavities 30, called through-cavities 30, passing through the substrate 10 between the first face 11 and the second face 12 of the substrate 10, the through-cavities 30 being spaced apart from each other by a portion of the substrate 10.

The cavities are formed, for example, by mechanical action (drilling) using a high-pressure water jet, or by photolithography followed by etching (chemical or dry). The cavities can also be formed by moulding, especially when the substrate 10 is a polydimethylsiloxane (PDMS) substrate.

The third step 403 consists in forming transparent electrodes 20 in the through-cavities 30, the transparent electrodes 20 passing through the substrate 10 from the first face 11 to the second face 12 of the substrate 10, and being electrically insulated from each other by the first electrically insulating and optically transparent material, precisely by the portion 14 of the substrate 10.

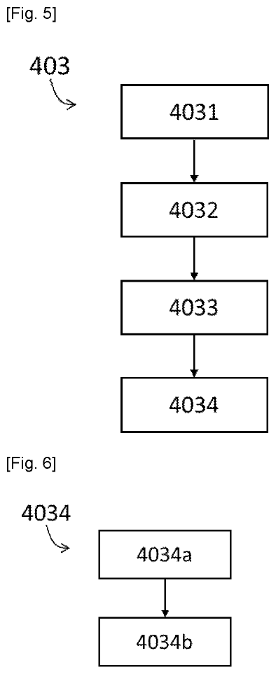

In an embodiment, the third step 403 comprises sub-steps 4031 to 4033. The sequence of these sub-steps is represented in FIG. 5.

FIGS. 7C to 7E represent a schematic cross-sectional view of these sub-steps 4031 to 4033.

With reference to FIG. 7C, the first sub-step 4031 of step 403 consists in depositing a first layer 40 of electrically conductive and optically transparent material onto:
the internal surface 31 of each of the through-cavities 30,
the first face 11 of the substrate 10, and
the second face 12 of the substrate 10.

In an embodiment, the deposition is carried out by virtue of a conformal deposition technique such as Atomic Layer Deposition (ALD). SnO₂, ZnO or AZO are examples of materials compatible with this type of deposition. In an embodiment, the electrically conductive and optically transparent material is therefore SnO₂, ZnO or AZO.

With reference to FIG. 7D, the second sub-step 4032 of step 403 consists of filling the through-cavities 30 with a transparent material 50 and forming an extra thickness 51 of the transparent material 50 on the first 11 and second 12 faces of the substrate.

The transparent material may be conductive or insulating. Examples of materials have been given previously in relation to FIGS. 1 and 2.

Filling the cavities 30 with the transparent material 50 may be carried out by capillary action or by suction. For example, the material 50 is dispensed onto the substrate 10 and a static pressure difference, due to the capillarity of the material 50 in the cavities 30 or to pumping (by means of a pump), enables the cavities 30 to be filled. Another filling method consists in depositing the substrate 10 onto a bath of transparent material 50 in an oven, and placing the oven in a vacuum and then at atmospheric pressure. Filling the cavities 30 with the transparent material 50 can also be carried out by screen printing or by localised deposition of droplets of the transparent material 50 onto the cavities 30 by ink jet or spray.

With reference to FIG. 7E, the third sub-step 4033 of step 403 consists in removing the extra thickness 51 of the transparent material 50 as well as the first layer 40 of electrically conductive and optically transparent material deposited onto the first and second faces 11-12 of the substrate 10.

This removal can be carried out by etching, for example by reactive ion etching (RIE) or by ion beam etching (IBE), or by chemical mechanical polishing (CMP).

At the end of sub-step 4033, a plurality of transparent, through-electrodes 20 have been made in the transparent, insulating substrate 10. Each electrode 20 comprises, inside the substrate, the hollow cylindrical portion 231 (formed by the remaining portion of the first layer 40) and the transparent core 232 disposed inside the hollow cylindrical portion 231 (formed by the material 50).

With reference to FIG. 4, the step 403 of forming the transparent through-electrodes 20 may also comprise an additional sub-step 4034, or fourth sub-step 4034, of forming, for each of the through-electrodes 20 formed, a first transparent electrode portion 21 on the first face 11 of the substrate 10 and a second transparent electrode portion 22 on the second face 12 of the substrate 10.

Sub-step 4034 comprises operations 4034a and 4034b, the sequence of which is represented in FIG. 6

The first operation 4034a, illustrated by FIG. 7F, consists in depositing a second layer 41 of electrically conductive and optically transparent material onto the first face 11 and the second face 12 of the substrate 10. The electrically conductive and optically transparent material forming the second layer 41 is beneficially SnO₂, but may be any type of conductive and optically transparent material (ITO, SnO₂, ZnO, AZO, etc.).

The second sub-step 4034b is represented in FIG. 7G.

The second operation 4034b of sub-step 4034, represented by FIG. 7G, consists in etching the second layer 41 of electrically conductive and optically transparent material into regions 60 of the first and second faces 11-12 of the substrate, the regions 60 being located around the transparent electrodes 20.

At the end of sub-step 4034, the through-electrodes 20 comprise, in addition to the internal portion 23 made in the preceding sub-steps 4031 and 4032, the first portion 21 and the second portion 22.

These two portions make it possible to fill the surface irregularities after the deposition sub-step 1032 and/or the removal sub-step 4033, such as hollows or voids forming on the first and second faces 11, 12 of the substrate 10. These irregularities are due to a capillary effect, and are observed in particular when the cavities have a high aspect ratio. This is especially the case when the substrate has a thickness E of 300 μm and the cavities have micrometric lateral dimensions.

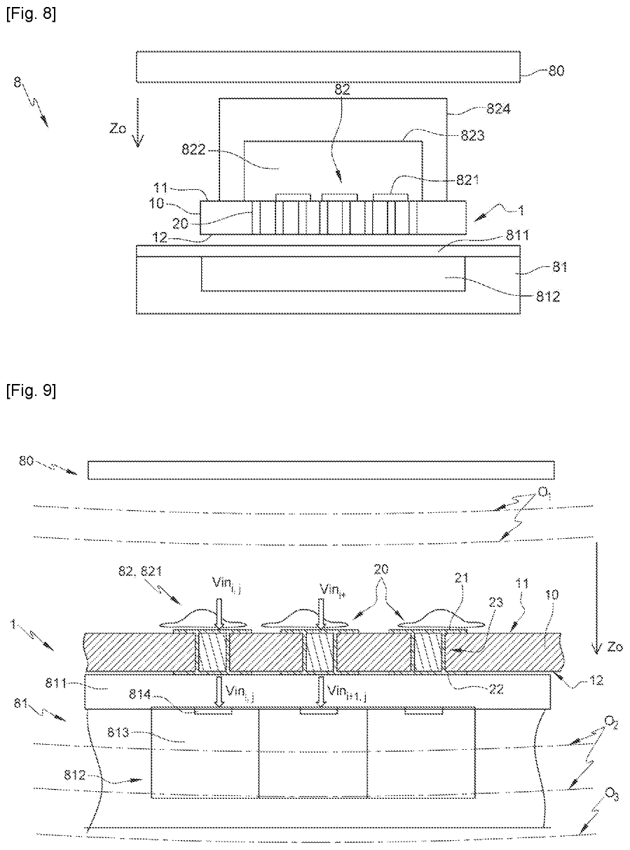

A third aspect of the invention relates to a system for analysing a sample, this system comprising the interconnection structure 1 described previously. FIG. 8 schematically represents an embodiment of this analysis system 8, ready to analyse a sample 82.

The sample 82 may be a biological sample which it is desired to characterise. It may be biological particles in a culture medium or in a body fluid. By biological particle, it is meant a cell, bacterium or other micro-organism with a size of between 1 μm and 500 μm.

The sample 82 is, for example, neuronal cells 821, or neurons, in a culture medium 822. Such cells are generally between 15 μm and 120 μm in size. They behave as electrical sources capable of producing an electrical signal, called the evoked potential or electrical signal Vin from the sample 82. The electrical signal Vin of the sample 82 is in the order of one mV or μV.

The system 8 is then intended to image the electrical activity of neurons with a spatial resolution at the scale of one neuron and a field of view in the order of one cm². The system 8 can thus be intended to understand certain neurodegenerative diseases such as Parkinson's or Alzheimer's by viewing neuronal electrical activity in real time and monitoring the effect of several treatments on this electrical activity in parallel.

The system 8 comprises a light source 80, the interconnection structure 1, and an electro-optical device 81.

FIG. 9 schematically represents an enlarged view of the interconnection structure 1 in the system 8 and the sample 82 disposed on the interconnection structure 1.

The light source 80 is capable of producing a light wave O1, called the incident light wave O1, in the direction of the sample 82, along a propagation direction Zo.

The light source 80 emits in the spectral band extending between 400 nm and 800 nm, called the visible band. In the case where the light source includes several elementary sources of the light-emitting diode (LED) or laser diode type, the elementary sources emit in the same visible band.

The light source 82 may comprise a diaphragm, a filter or a diffuser (not represented).

The light source 82 may comprise a polariser (not represented).

The interconnection structure 1 is disposed facing the light source 80.

The sample 82 is disposed on the first face 11 of the substrate 10 of the interconnection structure 1.

With reference to FIG. 8, the sample 82 is more particularly disposed as a 2D layer of neurons 821 on the first face 11 of the substrate 10. The neurons 821 are further disposed in the volume 823 of a fluidic chamber 824, the volume 823 of the fluidic chamber 824 being filled with a liquid, or culture medium 822, which may or may not be circulating and which makes it possible to culture the neurons 821. The fluidic chamber 824 is made of an optically transparent material, for example polydimethylsiloxane or PDMS. It has micrometric dimensions so that the interconnection structure 1 can receive several fluidic chambers.

The interconnection structure 1 is sufficiently rigid to receive the sample 82.

The electro-optical device 81 is disposed facing the second face 12 of the substrate 10 of the interconnection structure 1.

By electro-optical device, it is meant an optical device comprising at least one signal-controlled element having an electro-optical effect, this signal-controlled element being used to modulate a light beam passing through the device 81.

With reference to FIG. 9, the electro-optical device 81 may comprise an interface film 811, for example a polyamide layer, disposed between the interconnection structure 1 and the signal-controlled element 813 having an electro-optical effect.

The signal-controlled element 813 having an electro-optical effect is in an embodiment a liquid crystal. A liquid crystal has the property of modulating the polarisation of light passing through it under the effect of an electrical signal applied to it via a control electrode.

In the exemplary embodiment shown in FIGS. 8 and 9, the electro-optical device 81 is a liquid crystal cell 812, the control electrodes 814 of which are disposed facing the through-electrodes 20 of the interconnection structure 1. Each liquid crystal 813 of the liquid crystal cell 812 is controlled by the electrical signal Vin emitted by a cell 821 of the sample 82 and transmitted by the interconnection structure 1. Under the effect of this electrical signal Vin, the liquid crystal 813 locally modulates the light wave O2 transmitted by the sample and by the interconnection structure 1. The light wave locally modulated O₃ contains information about the electrical activity of the sample 82.

A light analyser comprising an image sensor and a polariser (not represented) may be used to record the image of the light wave transmitted and modulated O₃ by the system 8.

The electrical signal Vin from the sample is transmitted directly and faithfully, that is, respecting the spatial distribution of the electrical signal, through the interconnection structure 1, this by virtue of the through-electrodes 20 of the interconnection structure 1.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

It will be appreciated that the various embodiments and aspects of the inventions described previously are combinable according to any technically permissible combinations.

1. An interconnection structure comprising:
a substrate formed by a first electrically insulating and optically transparent material, the substrate comprising a first face and an opposite second face, the first face defining a plane of the substrate;
a plurality of transparent electrodes;
wherein the plurality of transparent electrodes pass through the substrate from the first face to the second face of the substrate in parallel to each other, and are electrically insulated from each other by the first electrically insulating and optically transparent material.

2. The interconnection structure according to claim 1, wherein each transparent electrode comprises a first portion disposed on the first face of the substrate.

3. The interconnection structure according to claim 2, wherein each transparent electrode further comprises a second portion disposed on the second face of the substrate facing the first portion.

4. The interconnection structure according to claim 3, wherein the transparent electrodes are symmetrical relative to a plane parallel to the plane of the substrate.

5. The interconnection structure according to claim 1, wherein each transparent electrode comprises a hollow cylindrical portion extending from the first face to the second face of the substrate, the interconnection structure further comprising a transparent core disposed inside the hollow cylindrical portion of each transparent electrode.

6. The interconnection structure according to claim 5, wherein the transparent core is formed by an electrically conductive and optically transparent material.

7. The interconnection structure according to claim 5, wherein the transparent core is formed by a second electrically insulating and optically transparent material.

8. The interconnection structure according to claim 5, wherein the hollow cylindrical portion has, in a plane parallel to the plane of the substrate, external dimensions of between 5 μm and 50 μm, and comprises a wall with a thickness of between 5 nm and 100 nm.

9. The interconnection structure according to claim 1, wherein the substrate has, between the first face and the second face of the substrate, a thickness of between 50 μm and 300 μm.

10. The interconnection structure according to claim 1, wherein the transparent electrodes are spaced apart from each other inside the substrate by an edge-to-edge distance of between 5 μm and 30 μm.

11. The interconnection structure according to claim 1, wherein the transparent electrodes are identical in size and shape and have, in the plane of the substrate, a first repeat pitch in a first direction and a second repeat pitch in a second direction intersecting the first direction.

12. The interconnection structure according to claim 1, wherein the transparent electrodes are formed by a transparent conductive polymer or a transparent conductive metal oxide such as tin dioxide.

13. A method for manufacturing an interconnection structure comprising:
providing a substrate formed by a first electrically insulating and optically transparent material, the substrate comprising a first face and an opposite second face, the first face defining a plane of the substrate;
arranging, in the substrate, a plurality of through-cavities, passing through the substrate between the first face and the second face of the substrate in parallel to each other, and being spaced apart from each other, and
forming, in the through-cavities, a plurality of transparent electrodes, said transparent electrodes passing through the substrate from the first face to the second face of the substrate in parallel to each other, and being electrically insulated from each other by the first electrically insulating and optically transparent material.

14. The method for manufacturing an interconnection structure according to claim 13, wherein forming the plurality of transparent electrodes comprises:
depositing a first layer of electrically conductive and optically transparent material onto:
the inner surface of each of the through-cavities,
the first face of the substrate, and
the second face of the substrate;
filling the through-cavities with a transparent material and forming an extra thickness of the transparent material on the first and second faces of the substrate, and
removing the extra thickness of the transparent material as well as portions of the first layer of electrically conductive and optically transparent material deposited onto the first face and the second face of the substrate.

15. The method for manufacturing an interconnection structure according to claim 14, wherein forming the transparent electrodes comprises further forming, for each of the through-electrodes formed, a first transparent electrode portion on the first face of the substrate and a second transparent electrode portion on the second face of the substrate, said further forming comprising:
depositing a second layer of electrically conductive and optically transparent material onto the first face and the second face of the substrate, and
etching the second layer of electrically conductive and optically transparent material in regions of the first and second faces of the substrate, the regions being located around the transparent electrodes formed.

16. A system for analysing a sample comprising:
a light source to produce an incident light wave,
an interconnection structure according to claim 1, the first face of the substrate being disposed facing the light source and being intended to receive a sample comprising at least one source adapted to produce an electrical signal of the sample, the electrical signal of the sample being transmitted from the first face of the substrate to the second face of the substrate by the through-electrode located facing said source,
an electro-optical device disposed facing the second face of the substrate,
the electro-optical device having a plurality of control electrodes disposed facing the plurality of through-electrodes of the interconnection structure, each control electrode of the plurality of control electrodes being connected to a through-electrode of the plurality of through-electrodes, so that the electro-optical device modulates the light wave transmitted by the sample under the effect of the electrical signal of the sample.

17. The analysis system according to claim 16, wherein the electro-optical device is a liquid crystal cell.
